(19) United States
(12) Patent Application Publication
(10) Pub. No.: US 2012/0265013 A1
(43) Pub. Date: Oct. 18, 2012

(54) PROPULSION ASSEMBLY FOR ENDOSCOPE

(76) Inventors: Shinichi YAMAKAWA, Kanagawa (JP); Tsuyoshi ASHIDA, Kanagawa (JP); Takayuki NAKAMURA, Kanagawa (JP); Yasunori OHTA, Kanagawa (JP)

(21) Appl. No.: 13/446,377

(22) Filed: Apr. 13, 2012

(30) Foreign Application Priority Data
Apr. 14, 2011 (JP) 2011-089972

(51) Int. Cl. A61B 1/00

(52) U.S. Cl. 600/114

(57) ABSTRACT

A propulsion assembly for an endoscope having a section of an elongated tube for entry in a tube of a body cavity is provided. The propulsion assembly includes a shaft sleeve, an endless track device, a support sleeve and a control wire. A first bevel gear is supported on the shaft sleeve, secured to a distal end portion of the control wire, for rotating about a first axis extending in an axial direction of the elongated tube upon rotation of the control wire. A second bevel gear is supported on the shaft sleeve in a rotatable manner about a second axis extending in a transverse direction of the elongated tube, meshed with the first bevel gear, engaged with the endless track device, for moving the endless track device in the axial direction. Preferably, the first bevel gear has a diameter smaller than a diameter of the second bevel gear.

### BACKGROUND OF THE INVENTION

1. Field of the Invention

The present invention relates to a propulsion assembly for an endoscope. More particularly, the present invention relates to a propulsion assembly for an endoscope, in which physical stress to a patient's body can be reduced effectively during imaging.

2. Description Related to the Prior Art

An endoscope includes a steering device and an elongated tube for entry in a tube of a body cavity of a patient. The steering device steers a head assembly in a direction as desired. Manipulation of the endoscope is a difficult process, because the large intestine is a tortuous organ in a human body, and some body parts are very changeable in the position in the body, for example, a sigmoid colon and transverse colon. If a doctor is insufficiently skilled in the manipulation, physical load to the body will be very large.

U.S. Pat. Nos. 6,971,990 and 7,736,300 (corresponding to JP-A 2009-513250) disclose a propulsion assembly for propelling an endoscope in an axial direction in a body cavity. The propulsion assembly includes an endless track device in a toroidal shape with an annular surface, for advancing the endoscope by turning the endless track device.

The propulsion assembly of the document includes a worm gear (worm thread or threaded sleeve) and a worm wheel (contact wheel). The worm gear is in a ring shape, supported around the elongated tube of the endoscope inside the endless track device, for rotating about the axial direction. The worm wheel is rotatable about an axis transverse to the axial direction of the endoscope, and caused to rotate by the worm gear for turning around the endless track device. The worm gear and the worm wheel are arranged in a radial direction which is transverse to the axial direction. Therefore, there is a problem in that the propulsion assembly has a large diameter. Physical stress to the patient's body during entry of the propulsion assembly is considerably large.

### SUMMARY OF THE INVENTION

In view of the foregoing problems, an object of the present invention is to provide a propulsion assembly for an endoscope, in which physical stress to a patient's body can be reduced effectively during imaging.

In order to achieve the above and other objects and advantages of this invention, a propulsion assembly for an endoscope having a section of an elongated tube for entry in a tube of a body cavity is provided. There is a shaft sleeve for mounting on the elongated tube. An endless track device is disposed around the shaft sleeve, for moving in an axial direction of the elongated tube in contacting an inner wall of the body cavity for propulsion. A support sleeve is contained in an inner space of the endless track device, for supporting the endless track device movably along inner and outer sleeve surfaces thereof. A control wire is disposed to extend along the elongated tube, having a proximal end portion caused to rotate by a drive source. A first gear is supported on the shaft sleeve, secured to a distal end portion of the control wire, for rotating about a first axis extending in the axial direction upon rotation of the control wire. A second gear is supported on the shaft sleeve in a rotatable manner about a second axis extending in a transverse direction of the elongated tube, meshed with the first gear, for driving the endless track device in the axial direction.

The first and second gears constitute a transmission device for transmitting turning of the control wire to the endless track device.

The first gear has first bevel gear teeth, and the second gear includes second bevel gear teeth meshed with the first bevel gear teeth, and plural engagement teeth for moving the endless track device.

In a preferred embodiment, furthermore, a contact wheel is formed coaxially with the second gear, for contacting and driving the endless track device.

The first and second gears are bevel gears.

The contact wheel has plural spur gear teeth.

The first gear has a diameter smaller than a diameter of the second gear.

The endless track device is in a toroidal shape with an annular surface.

The transmission device is constituted by plural transmission devices arranged about the axial direction outside the elongated tube.

In one preferred embodiment, the endless track device includes at least one endless belt.

The endless belt is constituted by plural endless belts, the transmission device is constituted by plural transmission devices, and the endless belts and the transmission devices are arranged about the axial direction outside the elongated tube.

Furthermore, an idler roller is supported on the support sleeve in a rotatable manner, for tensioning the endless track device in cooperation with the second gear.

The idler roller is constituted by two idler rollers arranged on proximal and distal sides from the second gear in the axial direction.

Furthermore, a pair of annular cover flanges are disposed on edges of respectively proximal and distal ends of the shaft sleeve, formed from flexible material, for closing between the endless track device and the elongated tube, and contacting the endless track device moving endlessly.

Furthermore, at least one auxiliary wheel is positioned beside the second gear in an offset manner about the axial direction, supported on the shaft sleeve in a rotatable manner about an axis extending in a transverse direction of the elongated tube, for engagement with the endless track device. An auxiliary transmission device transmits rotation of the second gear to the auxiliary wheel, to drive the endless track device in synchronism.

The transmission device is constituted by plural transmission devices arranged in the axial direction.

Furthermore, a pair of curved support surfaces are disposed at proximal and distal ends of the support sleeve, for supporting the endless track device movably.

In one preferred embodiment, furthermore, at least one pair of support rollers are secured to the proximal and distal ends of the support sleeve in a rotatable manner, for supporting the endless track device movably.

Accordingly, physical stress to a patient's body can be reduced effectively during imaging, because the first and second gears are so disposed as to reduce a diameter of the propulsion assembly.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a perspective view illustrating a head assembly of the endoscope and a propulsion assembly;

FIG. 3 is a vertical section illustrating the propulsion assembly;

FIG. 4 is an exploded perspective view illustrating a shaft sleeve, a support sleeve and a transmission device;

FIG. 5 is an exploded perspective view illustrating the shaft sleeve;

FIG. 6 is a perspective view illustrating a transmission device in one preferred propulsion assembly;

FIG. 7 is a vertical section illustrating another preferred propulsion assembly with plural sets of first and second gears arranged in the axial direction;

FIG. 8 is a vertical section illustrating still another preferred propulsion assembly having support rollers.

### DETAILED DESCRIPTION OF THE PREFERRED EMBODIMENT(S) OF THE PRESENT INVENTION

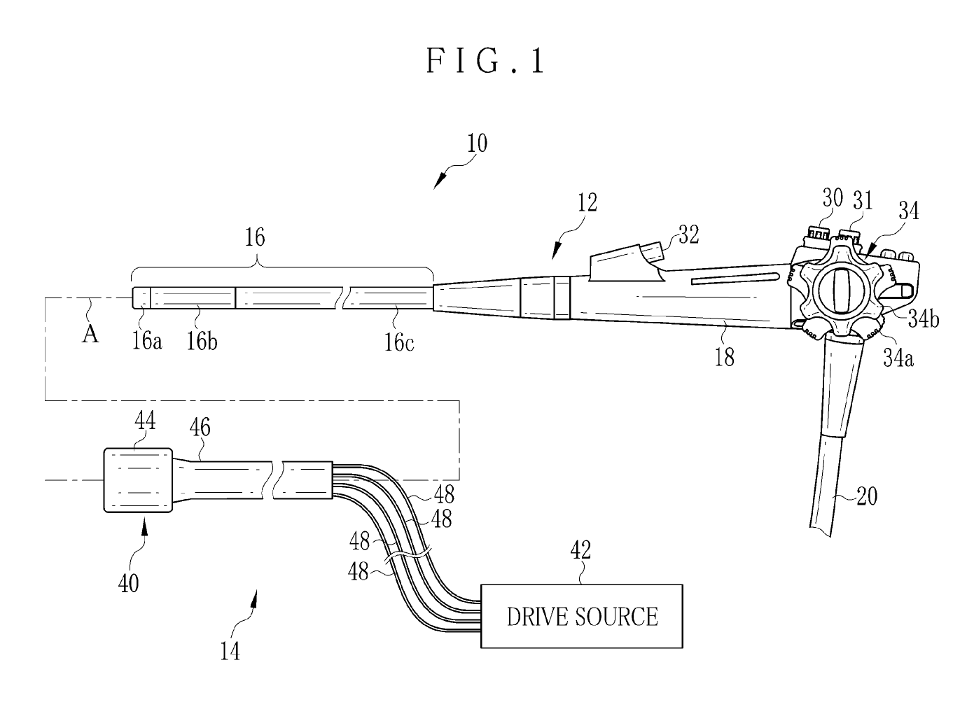

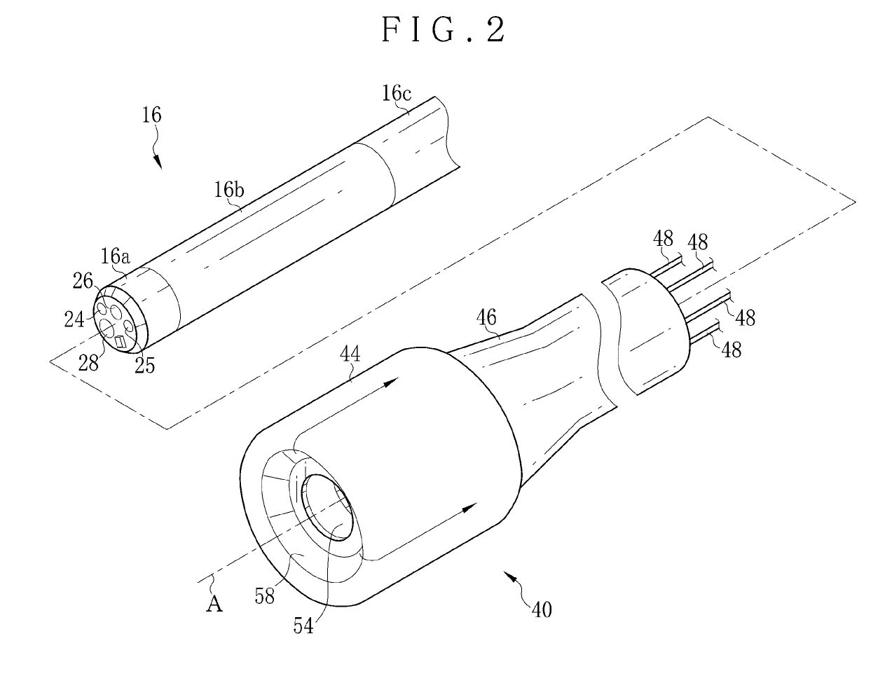

In FIGS. 1 and 2, an endoscope system 10 includes an electronic endoscope 12 and a propulsion assembly 14 for the endoscope 12. The endoscope 12 includes a section of an elongated tube 16 or guide tube, a handle device 18 and a universal cable 20. The elongated tube 16 is entered in a body cavity of a patient's body. The handle device 18 is disposed at a proximal end of the elongated tube 16. The universal cable 20 connects the handle device 18 to various external apparatuses in the endoscope system 10, such as a processing apparatus, light source apparatus, and fluid supply apparatus (all not shown).

The elongated tube 16 includes a head assembly 16a, a steering device 16b and a flexible device 16c arranged in a proximal direction. The head assembly 16a includes a lighting window 22, end nozzles 24 and 25, and a distal instrument opening 26. The lighting window 22 applies imaging light from the light source apparatus to an object of interest. The end nozzles 24 and 25 eject fluid from the fluid supply apparatus toward the imaging window, such as air and water. The distal instrument opening 26 is used for a tip of the electrocautery device to appear distally. Note that a proximal instrument opening 32 is formed in a proximal portion of the elongated tube 16, and initially receives entry of the electrocautery device toward the distal instrument opening 26.

An imaging window 28 is formed in the head assembly 16a and receives object light from an object of interest of a body cavity. A lens system and an image sensor are disposed behind the imaging window 28. Examples of the image sensor are a CCD and CMOS. There is a processing apparatus (not shown) to which the image sensor is connected by a signal cable, which extends through the elongated tube 16 and the handle device 18 with the universal cable 20. The processing apparatus drives the image sensor to image an object, and drives a monitor display panel (not shown) to display the object.

The steering device 16b is bendable, and is connected to the handle device 18 by wires or the like. The steering device 16b is steered up and down and to the right and left by the handle device 18, so as to orient the head assembly 16a in a desired direction. The flexible device 16c has as great a length as several meters for the head assembly 16a to reach an object of interest in the body cavity.

Fluid supply buttons 30 and 31 are disposed on the handle device 18 for supplying air and water through the end nozzle 24. The proximal instrument opening 32 is formed in the handle device 18 for entry of a medical instrument for treatment, such as an electrocautery device. A steering control unit 34 is incorporated in the handle device 18, and includes steering wheels 34a and 34b. When the steering wheel 34a is rotated, the steering device 16b is steered up or down. When the steering wheel 34b is rotated, the steering device 16b is steered to the right or left.

The propulsion assembly 14 is a guide assembly mounted on the endoscope 12 for assistance to forward and backward movement of the elongated tube 16. The propulsion assembly 14 includes a propulsion unit 40 and a drive source 42 having a motor. The propulsion unit 40 is entered in the body cavity. The drive source is disposed outside the body cavity, and controls the propulsion unit 40.

An endless track device 44 or a toroidal device is included in the propulsion unit 40. An example of material of the endless track device 44 is biocompatible plastic material having flexibility, for example, polyvinyl chloride, polyamide resin, fluorocarbon resin, polyurethane resin and the like. A support sleeve 52 is contained in the endless track device 44 and supports this in a movable manner in the axial direction A of the elongated tube 16. See FIG. 2. The endless track device 44 moves endlessly to propel the elongated tube 16 in the axial direction A.

An overtube 46 is connected with a proximal end of the propulsion unit 40, and is expandable and compressible in the axial direction A. A control wire 48 or torque wire extends through the overtube 46, and transmits driving force to the endless track device 44. A distal end of the control wire 48 is connected to the propulsion unit 40. A proximal end of the control wire 48 is connected to the drive source 42.

The drive source 42 includes a motor (not shown) and an input interface (not shown). The motor rotates the control wire 48. The input interface is operated manually to adjust a direction and speed of rotation of the motor. So a direction and speed of propulsion of the elongated tube 16 can be adjusted by control of the endless track device 44.

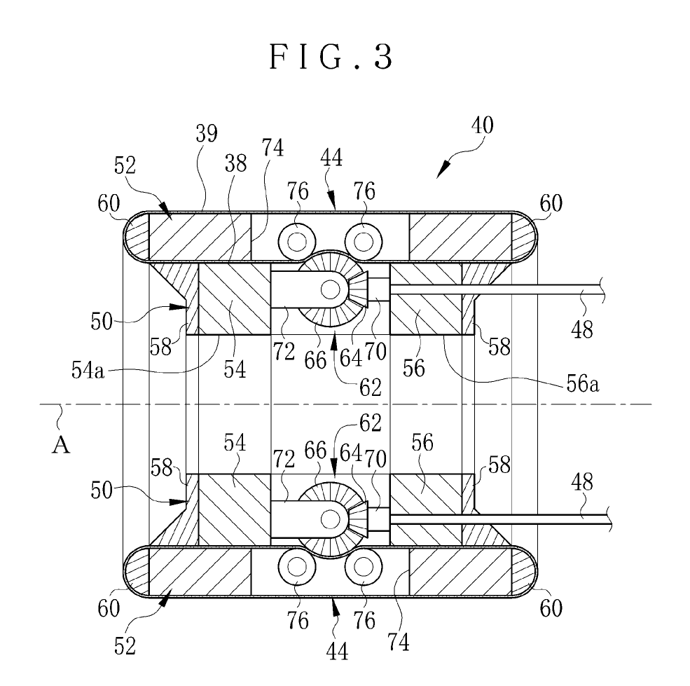

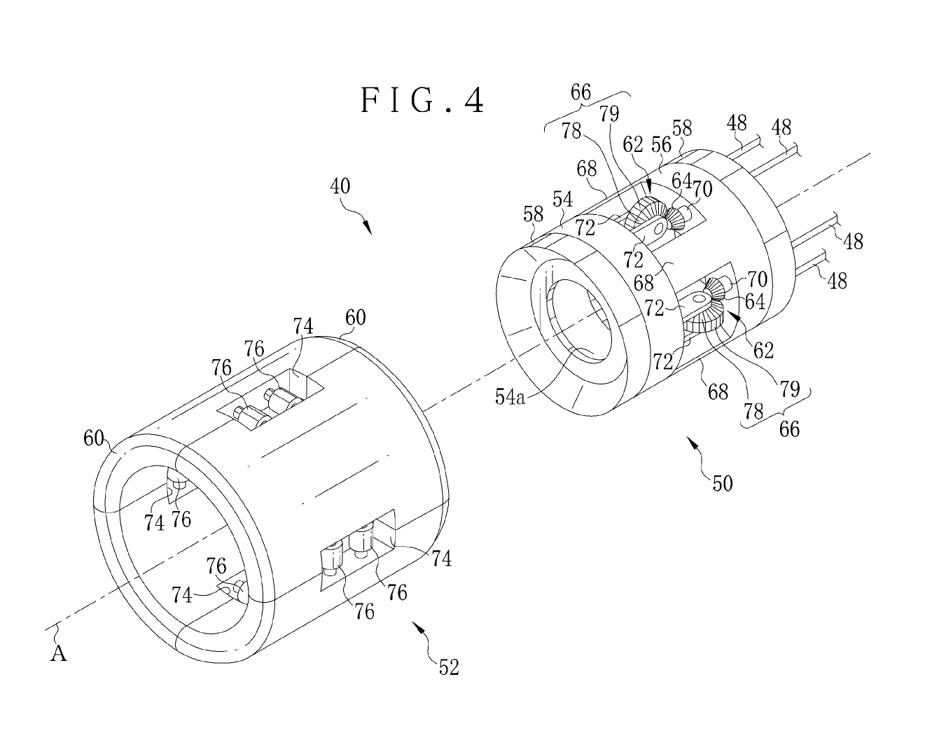

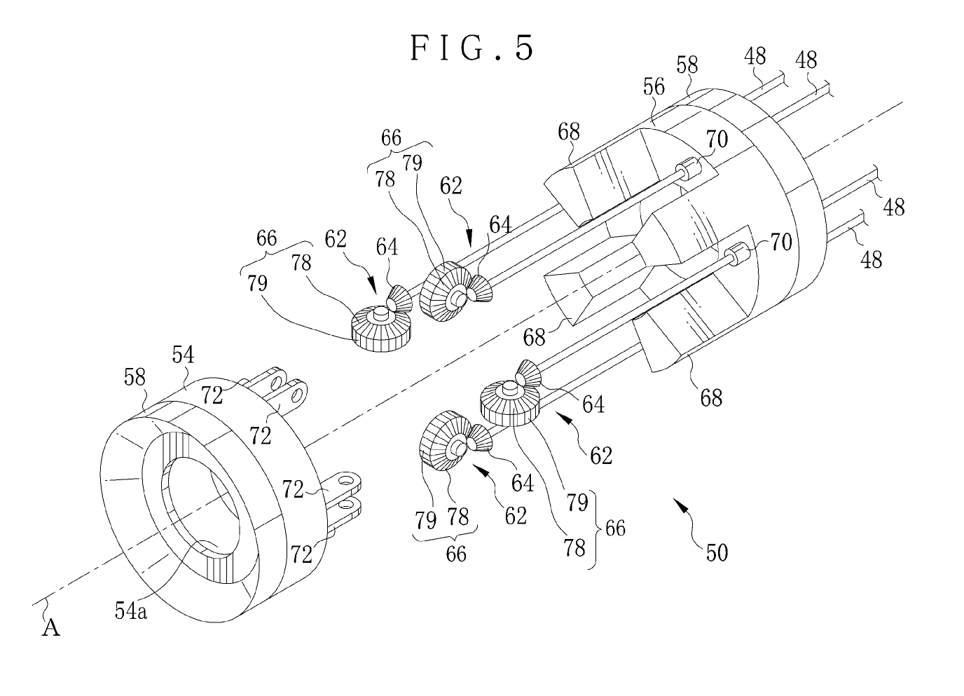

In FIGS. 3, 4 and 5, a shaft sleeve 50 is combined with the support sleeve 52 to constitute the propulsion unit 40. For the simplicity, the overtube 46 is not shown in FIG. 3. The overtube 46 and the endless track device 44 are not shown in FIGS. 4 and 5.

The shaft sleeve 50 includes a distal sleeve part 54 and a proximal sleeve part 56. A lumen 54a is defined in the distal sleeve part 54. A lumen 56a is defined in the proximal sleeve part 56. The elongated tube 16 is entered through the lumens 54a and 56a to mount the shaft sleeve 50 on the elongated tube 16. Also, the support sleeve 52 supports the endless track device 44 thereabout, and has a bore larger than an outer diameter of the shaft sleeve 50. The support sleeve 52 is disposed around the shaft sleeve 50.

A cover flange 58 or wiper flange is fitted on the shaft sleeve 50. A curved support surface 60 is formed on an end ring, which is fitted on the support sleeve 52 in a form of a vehicle bumper. The cover flange 58 is formed from a biocompatible plastic material with flexibility, and disposed annularly on each of proximal and distal ends of the shaft sleeve 50. The cover flange 58 has the annular shape for closing between the elongated tube 16 and a lower run 38 (return run) of the endless track device 44, and is pressed on the lower run 38. When the endless track device 44 turns around, the cover flange 58 frictionally contacts the lower run 38 and prevents incidental entry of foreign material between the lower run 38 and the elongated tube 16. The end ring having the curved support surface 60 is formed from material with a low coefficient of friction and high slip property, and disposed at each of proximal and distal ends of the support sleeve 52. The curved support surface 60 operates with slip even when its pressure to the endless track device 44 increases upon contact of an upper run 39 (active run) of the endless track device 44 on the wall of the body cavity.

A transmission device 62 or gear set or driving device is incorporated in the shaft sleeve 50, and includes a first bevel gear 64 and a second bevel gear 66 (spur bevel gear or roller gear) of a composite shape. The first and second bevel gears 64 and 66 are formed according to well-known techniques of the bevel gear. Tooth surfaces of the first and second bevel gears 64 and 66 are conical with an inclination to the gear axis. When the first bevel gear 64 rotates, the second bevel gear 66 rotates about an axis perpendicular to that of the first bevel gear 64. Four wall plates 68 project from the proximal sleeve part 56 in a distal direction toward the distal sleeve part 54. Four recesses are defined between the wall plates 68. The transmission device 62 with a set of the first and second bevel gears 64 and 66 is contained in each one of the recesses. There are four transmission devices 62, each of which includes the first and second bevel gears 64 and 66, and which are arranged about the axial direction A.

The first bevel gear 64 has bevel gear teeth disposed on a distal side. A proximal end portion of the first bevel gear 64 is fixedly secured to a distal end of the control wire 48. The control wire 48 extends along the elongated tube 16 for extracorporeal control. An annular spacer 70 is disposed on a distal end surface of the proximal sleeve part 56. The control wire 48 extends through the proximal sleeve part 56 and the annular spacer 70. When the control wire 48 rotates, the first bevel gear 64 rotates about the axial direction of the control wire 48 in front of the annular spacer 70.

A support bracket 72 or stay projects from a proximal surface of the distal sleeve part 54, and keeps the second bevel gear 66 rotatable about an axis which is perpendicular to the axial direction of the first bevel gear 64. Plural bevel gear teeth 78 are arranged in the second bevel gear 66, and meshed with teeth of the first bevel gear 64, and caused to rotate when the first bevel gear 64 rotates. A diameter of the second bevel gear 66 is larger than the first bevel gear 64. Also, the second bevel gear 66 has a contact wheel with plural spur gear teeth 79 projecting from an outer surface of the shaft sleeve 50.

An opening 74 is formed in the support sleeve 52 and disposed at the second bevel gear 66 (spur bevel gear or roller gear). A pair of idler rollers 76 are contained in the opening 74, and kept rotatable about an axis which is parallel to the axis of the second bevel gear 66. The idler rollers 76 are positioned on proximal and distal sides from the second bevel gear 66. The lower run 38 of the endless track device 44 is tensioned between a peripheral surface of the idler rollers 76 and the contact wheel (with the spur gear teeth 79) of the second bevel gear 66. When the second bevel gear 66 rotates, the endless track device 44 is caused to turn around endlessly, so as to rotate the idler rollers 76. Also, the support sleeve 52 is prevented by the idler rollers 76 from moving in the axial direction A both proximally and distally, and is positioned around the shaft sleeve 50.

As described heretofore, the bevel gears are used in the transmission device in the propulsion assembly 14. This feature is advantageous in reducing the outer diameter of the propulsion assembly 14 in comparison with the known technique in which a worm gear and a worm wheel are used in a transmission device. It is possible in the present invention to reduce physical stress to the body of the patient.

Note that details of the embodiment can be modified and are not limited to the above construction in which the first and second bevel gears 64 and 66 constitute the transmission device 62. For example, first and second gears in the transmission device 62 may be a face gear, hypoid gear, spur gear and the like in place of the bevel gears.

In the embodiment, the contact wheel of the second bevel gear 66 is the spur gear having the spur gear teeth 79 or engagement teeth. However, the contact wheel may have a pattern of numerous projections.

In the above embodiment, the four transmission devices are used to move the endless track device endlessly. However, the number of transmission devices for endlessly moving the endless track device may be three or less, or five or more.

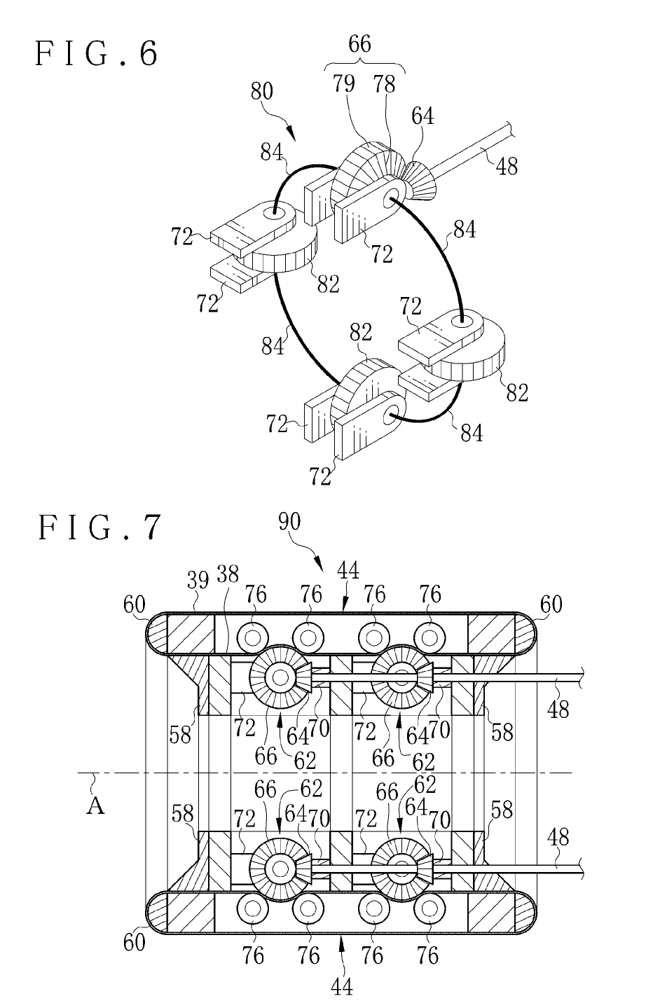

In FIG. 6, another preferred transmission device 80 for moving the endless track device 44 is illustrated. Elements similar to those of the above embodiment are designated with identical reference numerals.

In FIG. 6, the transmission device 80 includes only one set of the first and second bevel gears 64 and 66. Three auxiliary wheels 82 are associated with the transmission device 80 in place of the three remaining sets of the first and second bevel gears 64 and 66. The auxiliary wheels 82 have a diameter equal to that of the second bevel gear 66. The support bracket 72 keeps each of the auxiliary wheels 82 rotatable about an axis similar to the second bevel gear 66, so as to tension the endless track device 44 in cooperation with the idler rollers 76.

There is an auxiliary transmission device 84, such as a torque wire, universal joint and the like, for transmitting rotation of the second bevel gear 66 to the auxiliary wheels 82 in the transmission device 62. The auxiliary wheels 82 are rotated by rotation of the second bevel gear 66. This is effective in reducing the number of the first bevel gears 64 and the number of the torque wire in comparison with the above embodiment, so as to reduce the manufacturing cost. Note that the number of the auxiliary wheels 82 can be one or two or four or more to be rotated by the single second bevel gear 66.

In FIG. 7, another preferred propulsion assembly 90 is illustrated, in which two first bevel gears 64 are connected to the control wire 48 or torque wire. Two second bevel gears 66 are rotated by the first bevel gears 64. Furthermore, the number of the first bevel gears 64 can be three or more for the single control wire 48, to rotate three or more second bevel gears 66.

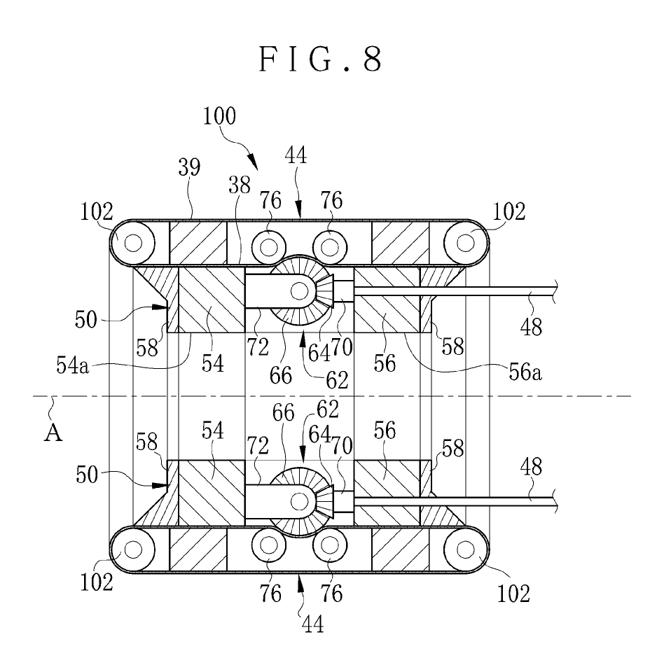

In FIG. 8, still another preferred propulsion assembly 100 is illustrated, and has rotatable support rollers 102 instead of the end rings with the curved support surface 60. Furthermore, the support rollers 102 can have a function similar to that of the curved support surface 60. The support rollers 102 can be formed from elastic material. Also, the support rollers 102 can be kept slidable in the proximal and distal directions by a sliding mechanism. A bias spring can be used to bias the support rollers 102 in the proximal or distal direction.

Note that the support rollers 102, although two pairs of the support rollers 102 are depicted in FIG. 8, can be a sufficiently high number of pairs of rollers, for example, four or six pairs. Thus, it is possible for the support rollers 102 to support the endless track device 44 in a form suitable for its annular shape.

In the above embodiment, the endless track device is in the toroidal form. However, endless belts may be used as endless track device. For example, four endless belts are arranged. Four transmission devices, which are similar to those of the above embodiment, drive respectively the endless belts. Furthermore, the feature of FIG. 6 may be used, in which the single transmission device drives the plural endless belts by transmission of rotation with the auxiliary wheels and control wire. Also, the feature of FIG. 7 can be combined with the use of the endless belts. In short, the two second bevel gears can drive each one of the endless belts. Also, the use of the support rollers 102 in FIG. 8 can be combined with the use of the endless belts.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly for an endoscope having a section of an elongated tube for entry in a tube of a body cavity, comprising:
a shaft sleeve for mounting on said elongated tube;
an endless track device, disposed around said shaft sleeve, for moving in an axial direction of said elongated tube in contacting an inner wall of said body cavity for propulsion;
a support sleeve, contained in an inner space of said endless track device, for supporting said endless track device movably along inner and outer sleeve surfaces thereof;
a control wire, disposed to extend along said elongated tube, having a proximal end portion caused to rotate by a drive source;
a first gear, supported on said shaft sleeve, secured to a distal end portion of said control wire, for rotating about a first axis extending in said axial direction upon rotation of said control wire;
a second gear, supported on said shaft sleeve in a rotatable manner about a second axis extending in a transverse direction of said elongated tube, meshed with said first gear, for driving said endless track device in said axial direction.

2. A propulsion assembly as defined in claim 1, wherein said first and second gears constitute a transmission device for transmitting turning of said control wire to said endless track device.

3. A propulsion assembly as defined in claim 2, wherein said first gear has first bevel gear teeth, and said second gear includes second bevel gear teeth meshed with said first bevel gear teeth, and plural engagement teeth for moving said endless track device.

4. A propulsion assembly as defined in claim 3, wherein said first gear has a diameter smaller than a diameter of said second gear.

5. A propulsion assembly as defined in claim 4, wherein said transmission device is constituted by plural transmission devices arranged about said shaft sleeve at a regular pitch.

6. A propulsion assembly as defined in claim 5, wherein said endless track device is in a toroidal shape with an annular surface.

7. A propulsion assembly as defined in claim 5, wherein said endless track device includes endless belts associated with respectively said transmission devices.

8. A propulsion assembly as defined in claim 3, further comprising at least one idler roller, supported on said support sleeve in a rotatable manner, for tensioning said endless track device in cooperation with said second gear.

9. A propulsion assembly as defined in claim 8, wherein said at least one idler roller is constituted by two idler rollers arranged on proximal and distal sides from said second gear in said axial direction.

10. A propulsion assembly as defined in claim 3, further comprising a pair of annular cover flanges, disposed on edges of respectively proximal and distal ends of said shaft sleeve, formed from flexible material, for closing between said endless track device and said elongated tube in a movable condition of said endless track device.

11. A propulsion assembly as defined in claim 3, further comprising:
at least one auxiliary wheel, positioned beside said second gear in an offset manner about said axial direction, supported on said shaft sleeve in a rotatable manner about an axis extending in a transverse direction of said elongated tube, for engagement with said endless track device;
an auxiliary transmission device for transmitting rotation of said second gear to said auxiliary wheel, to drive said endless track device with said second gear.

12. A propulsion assembly as defined in claim 2, wherein said transmission device is constituted by plural transmission devices arranged in said axial direction.

13. A propulsion assembly as defined in claim 3, further comprising a pair of curved support surfaces, disposed at proximal and distal ends of said support sleeve, for supporting said endless track device movably.

14. A propulsion assembly as defined in claim 3, further comprising at least one pair of support rollers, secured to said proximal and distal ends of said support sleeve in a rotatable manner, for supporting said endless track device movably.
